(19) United States
(12) Patent Application Publication
(10) Pub. No.: US 2022/0273434 A1
(43) Pub. Date: Sep. 1, 2022

(54) DEVICE FOR TRANSCATHETER GRABBING AND SECURING A NATIVE MITRAL VALVE LEAFLET TO A PROSTHESIS

(71) Applicant: THE REGENTS OF THE UNIVERSITY OF CALIFORNIA, Oakland, CA (US)

(72) Inventors: Arash Kheradvar, Irvine, CA (US); Daryl C. Nguyen, Irvine, CA (US)

(21) Appl. No.: 17/680,636

(22) Filed: Feb. 25, 2022

Related U.S. Application Data
(60) Provisional application No. 63/154,080, filed on Feb. 26, 2021.

(51) Int. Cl. A61F 2/24

(52) U.S. Cl. CPC A61F 2/2457; A61F 2220/0016

(57) ABSTRACT

The present invention is directed to a device for first grabbing, and then securing the native atrioventricular valve's leaflet to a prosthesis via transcatheter means. The present invention features a grasping device for securing a leaflet component of a native mitral valve. The leaflet component comprises a leaflet and chordae tendineae associated with the. In some embodiments, the device may comprise a first grasping component configured to grab chordae tendineae of said native mitral leaflet component, and a second grasping component configured to anchor the native mitral leaflet component to a prosthetic component. In some embodiments, the first grasping component may comprise at least one grasping arm configured to transition from a non-extended state to an extended state to a grasping state. In some embodiments, the second grasping component may comprise a hook disposed at a distal end of the device capable of anchoring to the prosthetic component.

### CROSS-REFERENCES TO RELATED APPLICATIONS

This application is a non-provisional and claims benefit of U.S. Provisional Application No. 63/154,080 filed Feb. 26, 2021, the specification of which is incorporated herein in their entirety by reference

### FIELD OF THE INVENTION

The present invention is directed to a device for first grabbing, and then securing the native atrioventricular valve's leaflet to a prosthesis via transcatheter means.

### BACKGROUND OF THE INVENTION

Left ventricular outflow tract (LVOT) obstruction is a leading cause of mortality and exclusion from transcatheter mitral valve replacement (TMVR). LVOT obstruction is the most frightening TMVR complication, which occurs in up to 40% of valves implanted in a native mitral annular calcification, 5% of valve-in-ring, and 2% of valve-in-valve cases (Yoon SH, Whisenant BK, Bleiziffer S, et al. Outcomes of transcatheter mitral valve replacement for degenerated bioprostheses, failed annuloplasty rings, and mitral annular calcification. Eur Heart J 2019;40:441-51.), and leads to 62% in-hospital mortality (Guerrero M, Urena M, Himbert D, et al. 1-year outcomes of transcatheter mitral valve replacement in patients with severe mitral annular calcification. J Am Coll Cardiol 2018;71:1841-53.). Potential for LVOT obstruction is the most important cause for patient exclusion for TMVR, with 49% of the patients for valves implants in a native mitral annular calcification and 6% for valve-in-ring excluded for TMVR due to predicted risk of LVOT obstruction (Khan JM, et al. Anterior Leaflet Laceration to Prevent Ventricular Outflow Tract Obstruction During Transcatheter Mitral Valve Replacement, J Am Coll Cardiol. 2019 May 28; 73(20): 2521-2534.).

There are currently two mechanisms that lead to TMVR-induced LVOT obstruction. (1) static obstruction occurs when the native mitral valve's anterior leaflet is pushed toward the interventricular septum by the mitral valve prosthesis, creating a narrowed and elongated “neo-LVOT” (Blanke P, Naoum C, Dvir D, et al. Predicting LVOT obstruction in transcatheter mitral valve implantation: concept of the neo-LVOT. J Am Coll Cardiol Img 2017;10:482-5). (2) Dynamic obstruction occurs when the narrowed neo-LVOT generates Bernoulli forces that pull the anterior mitral leaflet toward the interventricular septum during systole (Khan JM, Trivedi U, Gomes A, Lederman RJ, Hildick-Smith D. ‘Rescue’ LAMPOON to treat transcatheter mitral valve replacement-associated left ventricular outflow tract obstruction. J Am Coll Cardiol Intv 2019. Feb. 7). A long anterior mitral leaflet with redundant chordae could be a risk factor (Khan JM, Lederman RJ, Devireddy CM, et al. LAMPOON to facilitate Tendyne transcatheter mitral valve replacement. J Am Coll Cardiol Intv 2018;11:2014-7.), which may also prolapse back into the transcatheterly-implanted mitral valve, interfering with valve closure and causing acute valve failure (Greenbaum AB, Condado JF, Eng M, et al. Long or redundant leaflet complicating transcatheter mitral valve replacement: case vignettes that advocate for removal or reduction of the anterior mitral leaflet. Catheter Cardiovasc Intery 2018;92: 627-32). A solution to avoid both static and dynamic TMVR-induced LVOT obstruction is first grabbing and then properly securing the native mitral valve's anterior leaflet to the implanted mitral prosthesis. The present invention comprises an assembly of components that work together as a device for first grabbing, and then securing the native mitral valve's leaflet to a mitral prosthesis via transcatheter means. The following shows step-by-step how a device mechanism through a catheter that transseptally (entering through the left atrium and then left ventricle through the interatrial septum) passes or directly enters the left atrium and then the left ventricle to grab the mitral anterior leaflet and then secure it to the mitral prosthesis.

### BRIEF SUMMARY OF THE INVENTION

It is an objective of the present invention to provide devices and methods that allow for first grabbing, and then securing the native atrioventricular valve's leaflet to a prosthesis via transcatheter means, as specified in the independent claims. Embodiments of the invention are given in the dependent claims. Embodiments of the present invention can be freely combined with each other if they are not mutually exclusive.

The present invention features a grasping device for securing a mitral leaflet component of a native mitral valve. The mitral leaflet component comprises a leaflet and chordae tendineae associated with said leaflet. In some embodiments, the device may comprise a first grasping component configured to grab chordae tendineae of said mitral leaflet component, and a second grasping component configured to anchor the mitral leaflet component to a prosthetic component. In some embodiments, the first grasping component may comprise at least one grasping arm configured to transition from a non-extended state to an extended state to a grasping state. In some embodiments, the second grasping component may comprise a hook disposed at a distal end of the device capable of anchoring to the prosthetic component. In some embodiments, the prosthetic component comprises a stent.

The present invention features a method for securing a mitral leaflet component of a native mitral valve. The mitral leaflet component comprises a leaflet and chordae tendineae associated with the leaflet. In some embodiments, the method may comprise providing a grasping device comprising a first grasping component configured to grab chordae tendineae of said mitral leaflet component, and a second grasping component configured to anchor the mitral leaflet component to a prosthetic component. The method may further comprise positioning the device to a side of chordae tendineae of the mitral leaflet component. Positioning the device to the side of chordae tendineae may comprise deploying the device into the mitral valve through a catheter. The method may further comprise actuating the first grasping component to grab chordae tendineae of the mitral leaflet component. The method may further comprise actuating the second grasping component to anchor the mitral leaflet component to the prosthetic component. In some embodiments, the method may further comprise removing the catheter from the mitral valve, leaving the device in place. In some embodiments, the first grasping component of the device may comprise at least a grasping arm configured to transition from a non-extended state to an extended state to a grasping state. In some embodiments, the second grasping component of the device may comprise a hook disposed at a distal end of the device. In some embodiments, the prosthetic component comprises a stent.

One of the unique and inventive technical features of the present invention is the use of a first grasping component for grasping chordae tendineae, and a second grasping component for anchoring to a prosthetic component in order to immobilize an anterior leaflet component AFTER grasping chordae tendineae. Without wishing to limit the invention to any theory or mechanism, it is believed that the technical feature of the present invention advantageously provides for the ability to prevent LVOT obstruction to a pre-deployed prosthetic component without risking damage to chordae tendineae even in the case of user error. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A-1B show generic schematics of the device for grabbing onto chordae tendineae of a mitral leaflet component and anchoring to a prosthetic component.

FIG. 2 shows a method of a device comprising a first grasping component for grabbing onto chordae tendineae of a mitral leaflet component.

FIG. 3A-3L show a method of deploying a transcatheter device for securing a mitral leaflet component of a native mitral valve, actuating a first grasping component of the device to grab onto chordae tendineae of a mitral leaflet component, actuating a second grasping component to anchor to a prosthetic component, and removing the catheter leaving the device in place.

FIG. 4A-4D shows an alternate embodiment of a method of the device comprising a first grasping component for grabbing onto chordae tendineae of a mitral leaflet component and a second grasping component for anchoring to a prosthetic component.

### DETAILED DESCRIPTION OF THE INVENTION

Following is a list of elements corresponding to a particular element referred to herein:

1 chordae tendineae

100 catheter

200 grasping device

210 first grasping component

215 memory wire

220 first slide lock

225 second slide lock

230 second grasping component

300 prosthetic component

410 chordae hook actuator

415 first detachment region

420 prosthesis hook actuator

425 second detachment region

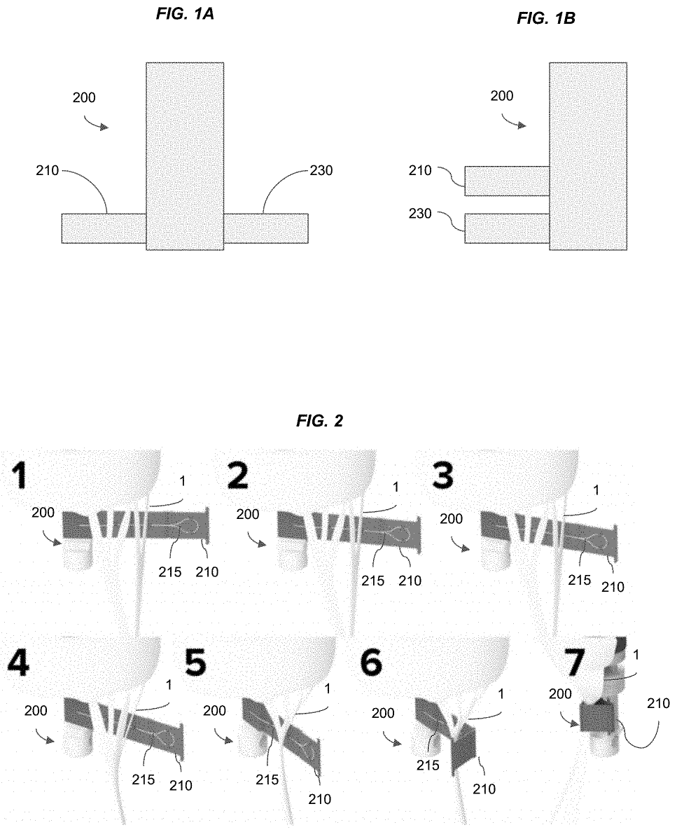

Referring now to FIG. 1A-1B, the present invention features a grasping device (200) for securing a leaflet component of a native atrioventricular valve. In some embodiments, the present invention features a grasping device (200) for securing a leaflet component of a native mitral valve (i.e., a bicuspid valve). In other embodiments, the present invention features a grasping device (200) for securing a leaflet component of a native tricuspid valve.

The leaflet component may comprise a leaflet and a chordae tendineae (1) associated with said leaflet. In some embodiments, the leaflet component comprises a mitral leaflet component. In other embodiments, the leaflet component comprises a tricuspid leaflet component.

The mitral leaflet component comprises a leaflet (e.g., an anterior leaflet or a posterior leaflet) and chordae tendineae (1) associated with said leaflet. In some embodiments, the device (200) may comprise a first grasping component (210) configured to grab chordae tendineae (1) of said mitral leaflet component, and a second grasping component (230) configured to anchor the mitral leaflet component to a prosthetic component (300). In some embodiments, the prosthetic component (300) is a mitral prosthetic component (300) (e.g., a stent).

As shown in FIG. 1A, in one embodiment, the first grasping component (210) and the second grasping component (230) of the device (200) may be disposed on opposite sides of the device (200). As shown in FIG. 1B, in another embodiment, the first grasping component (210) and the second grasping component (230) may be disposed on the same side of the device (200), or in any other configuration of positions on the device (200).

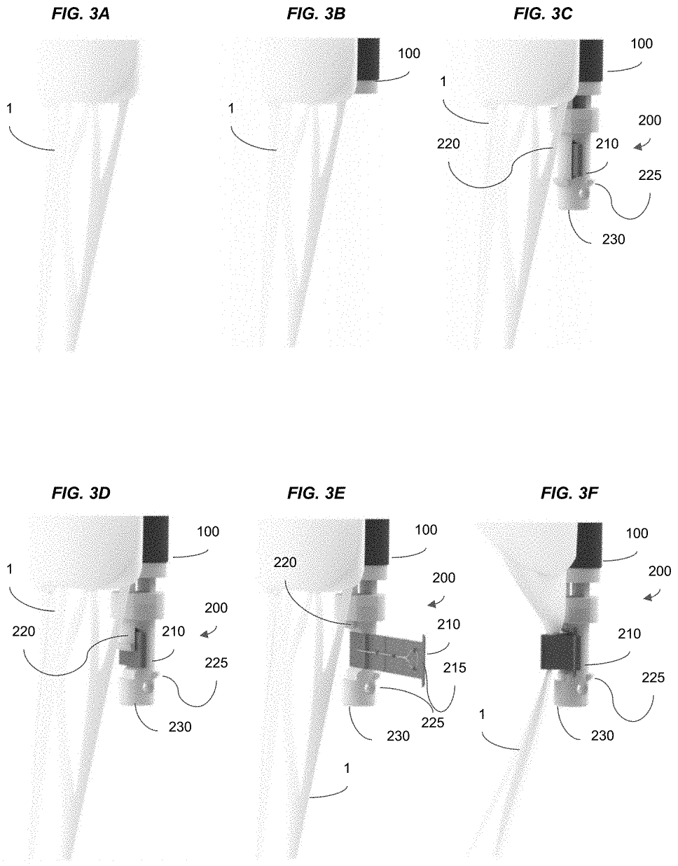

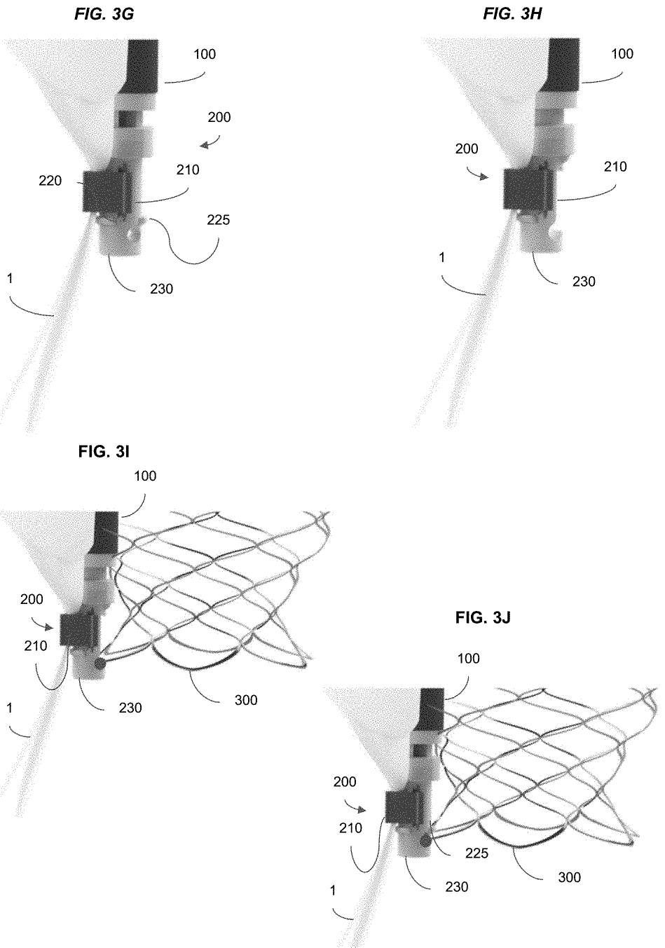

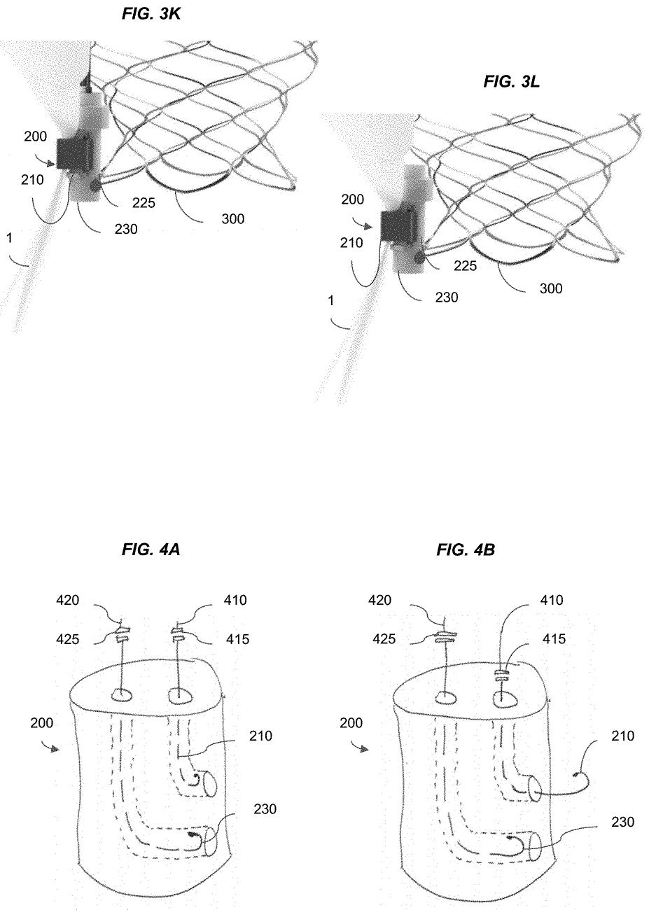

Referring now to FIG. 2 and FIG. 3A-3L, in some embodiments, the first grasping component (210) may comprise at least one grasping arm comprising at least one memory wire (215) disposed lengthwise and attached to a surface of the arm. In other embodiments, the first grasping component (210) may comprise a plurality of grasping arms, each comprising at least one memory wire (215) disposed lengthwise and attached to a surface of said arm. In some embodiments, the plurality of grasping arms are stacked on top of each other (e.g., vertically stacked). In certain embodiments, the height of each arm can be adjusted (e.g., minimized) such that the plurality of grasping arms can fit into the same space as one grasping arm. For devices (200) comprising a plurality of grasping arms a mechanism may be used to spring the grasping arms open in a radial pattern (i.e., the plurality of grasping arms may be radially symmetrical). The mechanism may be further used to lock the plurality of grasping arms in a secure position, thus keeping the leaflet component (e.g., the mitral leaflet component comprising a leaflet and a chordae tendineae) contained.

In some embodiments, the memory wire (215) is disposed on an inner surface of a grasping arm. In other embodiments, the memory wire (215) is disposed on an exterior surface of a grasping arm. The surface of the grasping arm in which the memory wire (215) is disposed will fold inward. For example, if the memory wire (215) is disposed on the inner surface of the grasping arm the inner surface will fold inward (see FIG. 2 and FIG. 3A-3L).

The arm may be configured to transition from a non-extended state to an extended state to a grasping state. In some embodiments, transitioning from the extended state to the grasping state comprises pulling the memory wire (215) to curl the arm (210) such that the first end meets or overlaps the second end of the arm (210). The memory wire (215) may extend from the surface of the arm to a point that allows a user to interact with the memory wire (215).

In some embodiments, the device (200) may further comprise a first slide lock (220) adapted to slide between a locked position and an unlocked position along the device (200). In other embodiments, the device (200) may further comprise a plurality of first slide locks (220) adapted to slide between a locked position and an unlocked position along the device (200). For example, a plurality of first slide locks (220) may be used with devices (200) described herein comprising a plurality of grasping arms.

The unlocked position of the first slide lock (220) may permit the arm (210) to transition from the non-extended state to the extended state. The locked position of the first slide lock (220) may keep the arm in the non-extended state or the grasping state, depending on which state the arm is currently in. An actuation method of the first slide lock (220) may be selected from a group comprising pulling a wire (e.g., an external wire) operatively coupled to the first slide lock (220) and twisting a wire coil operatively coupled to the first slide lock (220). A resting state of the memory wire (215) may be a straight line, and the arm may be transitioned from the non-extended state to the extended state by allowing the memory wire (215) to extend into the straight line.

In some embodiments, the memory wire (215) may be compressed in the non-extended (e.g., a folded) state and may remain compressed until exposed to body temperature. In some embodiments, once inside the body (i.e., once the memory wire (215) reaches body temperature) the memory wire (215) will want to extend into a straight line, thus extending the grasping arm; however, the first slide lock (220) prevents the grasping arm from extending into a straight line. Then where the first slide lock (220) is released, the grasping arm transitions into an extending state (e.g., a straight line). Once the grasping arm has captured the mitral leaflet component (e.g, chordae tendineae (1)), externally pulling the memory wire (215) will cause the grasping arm(s) to fold as a triangle (see FIG. 3F). In some embodiments, the grasping arms are held in the triangular position to minimize squishing the mitral leaflet component (e.g, chordae tendineae (1)) like a flat fold would.

In some embodiments, the memory wire (215) comprises a nitinol wire. In other embodiments, the memory wire (215) comprises elastic nitinol. In further embodiments, the memory wire (215) comprises a memory shape alloy.

In some embodiments, the wire (e.g., a nitinol wire, an elastic nitinol wire, or a memory shape alloy) is operatively coupled to the memory wire (215). The external pulling of the memory wire will force the grasping arm to close (pulling force needs to overcome the force that keeps the memory wanting to keep the arm extended).

The arm may further comprise a set of teeth disposed at the first end for locking the arm in the grasping state. In some embodiments, the set of teeth prevents sliding of the grasping arm from the grasping state. For example, when the grasping arm is ready to be locked into the grasping state, there is nothing keeping the grasping arm from unfolding and sliding out of the gate. Therefore, the set of teeth prevents the grasping arm from unfolding into the extended state. In some embodiments, the set of teeth is taller/longer than the height of the gate hole. In some embodiments, the set of teeth comprises stiff biocompatible materials including but not limited to metal or plastic.

In some embodiments, the second grasping component (230) may comprise a prosthesis grasping hook disposed at a distal end of the device (200) capable of anchoring to the prosthetic component. In some embodiments, the second grasping component (230) anchors the mitral leaflet component to the prosthetic component (300). Specifically, the second grasping component (230) may anchor a mitral valve leaflet (e.g., the anterior leaflet of the mitral valve). Without wishing to limit the present invention to any theory or mechanism it is believed that anchoring of the anterior leaflet of the mitral valve will prevent LVOT obstruction.

In some embodiments, the device (200) may further comprise a second slide lock (225) adapted to slide between a locked position and an unlocked position along the device (200), wherein the locked position closes the prosthesis grasping hook, and wherein the unlocked position allows the prosthesis grasping hook to anchor the mitral leaflet component to the prosthetic component (300). An actuation method of the second slide lock (225) may be selected from a group comprising pulling a wire operatively coupled to the second slide lock (225) and twisting a wire coil operatively coupled to the second slide lock (225). In some embodiments, the prosthetic component (300) comprises a stent.

Referring now to FIGS. 2 and 3A-3L, the present invention features a method for securing a leaflet component of a native mitral valve. In some embodiments, the leaflet component comprises a mitral leaflet component. The mitral leaflet component comprises a leaflet and chordae tendineae (1) associated with said leaflet. In some embodiments, the method may comprise providing a grasping device (200) comprising a first grasping component (210) configured to grab chordae tendineae (1) of said mitral leaflet component, and a second grasping component (230) configured to anchor the mitral leaflet component to a prosthetic component (300). In some embodiments, the prosthetic component (300) is a mitral prosthetic component (300). The method may further comprise positioning the device (200) to a side of chordae tendineae (1) of the mitral leaflet component (see FIGS. 3A-3C). Positioning the device (200) to the side of chordae tendineae (1) may comprise deploying the device (200) into the mitral valve through a delivery system (e.g., a catheter (100)).

In some embodiments, the delivery system (e.g., a catheter (100)) for deploying the device (200) may be able to rotate to move the grasping arms into an ideal location. An ideal position to start may be right before a leaflet of the mitral leaflet component (e.g., an anterior leaflet) so that the arm will grab chordae tendineae (1) through the sweep. In some embodiments, the device (200) may be rotated into an ideal location (e.g., a position in which the chordae tendineae (1) may be grabbed) regardless of the starting position.

The method may further comprise actuating the first grasping component (210) to grab chordae tendineae (1) of the mitral leaflet component (see FIG. 2). The method may further comprise actuating the second grasping component (230) to anchor the mitral leaflet component to the prosthetic component (300) (see FIG. 3I). In some embodiments, the method further comprises actuating the second grasping component (230) to anchor a mitral leaflet (e.g., an anterior leaflet) to the prosthetic component (300) (see FIG. 3I). In some embodiments, the method may further comprise removing the catheter from the left ventricle, leaving the device (200) in place (see FIGS. 3K-3L).

In some embodiments, the same delivery system is used to deploy the devices (200) described herein and the prosthetic component (300). In other embodiments, the same catheter (i.e., delivery catheter) is used to deploy the devices (200) described herein and the prosthetic component (300).

In some embodiments, the devices (200) described herein are permanent devices. In other embodiments, the device (200) described herein are semi-permanent devices.

In some embodiments, the first grasping component (210) of the device (200) may comprise at least one grasping arm comprising a memory wire (215) disposed lengthwise and attached to a surface of the arm. In some embodiments, the arm may be configured to transition from a non-extended state to an extended state to a grasping state. The method may further comprise pulling the memory wire (215) to curl the arm such that the first end meets or overlaps the second end of the arm in order to transition from the extended state to the grasping state.

In some embodiments, the device (200) may further comprise a first slide lock (220) adapted to slide between a locked position and an unlocked position along the device (200). The unlocked position of the first slide lock (220) may permit the arm to transition from the non-extended state to the extended state. The locked position of the first slide lock (220) may keep the arm in the non-extended state or the grasping state, depending on which state the arm is currently in. In some embodiments, the method may further comprise sliding the first slide lock (220) into the unlocked position to permit the arm to transition from the non-extended state to the extended state (see FIGS. 3D-3E), and sliding the first slide lock (220) into the locked position to keep the arm in the grasping state (see FIGS. 3F-3G). An actuation method of the first slide lock (220) is selected from a group comprising pulling a wire operatively coupled to the first slide lock (220) and twisting a wire coil operatively coupled to the first slide lock (220). In some embodiments, a resting state of the memory wire (215) may be a straight line, and the arm may be transitioned from the non-extended state to the extended state by allowing the memory wire (215) to extend into the straight line. In some embodiments, the memory wire (215) may comprise a nitinol wire. In some embodiments, the arm may further comprise a set of teeth disposed at the first end for locking the arm (210) in the grasping state.

In some embodiments, the second grasping component (230) of the device (200) may comprise a prosthesis grasping hook disposed at a distal end of the device (200). In some embodiments, the device (200) may further comprise a second slide lock (225) adapted to slide between a locked position and an unlocked position along the device (200). The locked position may close the prosthesis grasping hook, and the unlocked position may allow the prosthesis grasping hook to anchor the mitral leaflet component to the prosthetic component (300). In some embodiments, the method may further comprise sliding the second slide lock (225) into the unlocked position to allow the prosthesis grasping hook to anchor the mitral leaflet component to the prosthetic component (300) (see FIGS. 3H-3I), and sliding the second slide lock (225) into the locked position to lock the mitral leaflet component to the prosthetic component (300) (see FIG. 3J). An actuation method of the second slide lock (225) may be selected from a group comprising pulling a wire operatively coupled to the second slide lock (225) and twisting a wire coil operatively coupled to the second slide lock (225). In some embodiments, the prosthetic component (300) comprises a stent. In some embodiments, the method may comprise positioning the device (200) at a side of chordae tendineae (1) near papillary muscles associated with chordae tendineae (1), and sliding the device up chordae tendineae (1) towards the mitral leaflet component.

In some embodiments, the prosthetic component (300) comprises a stent. In some embodiments, the stent further comprises a heart valve. In other embodiments, the prosthetic component (300) comprises a valve (e.g., a heart valve). In some embodiments, the prosthetic component (300) may comprise a landing base which aids in the deployment of the prosthetic component (300) (e.g., a prosthetic component (300) comprising a stent).

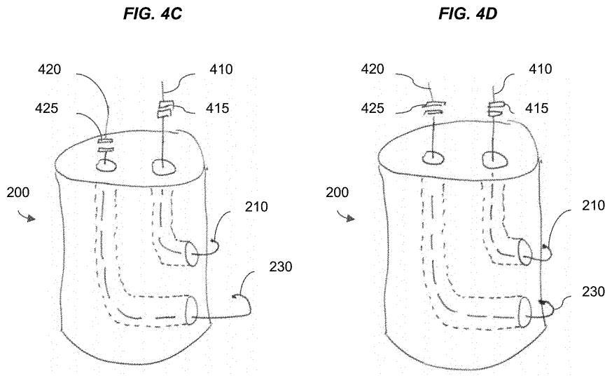

Referring now to FIGS. 4A-4D, in other embodiments, the first grasping component (210) of the device may comprise a chordae grasping hook. The chordae grasping hook may further comprise a chordae hook actuator (410) and a first detachment region (415). The chordae grasping hook may be actuated by the chordae hook actuator (410). Actuating the chordae hook actuator (410) may comprise pulling, twisting, pushing, or in any way moving the chordae hook actuator (410). The chordae grasping hook, upon grabbing chordae tendineae (1) of the mitral leaflet component, may detach from the chordae hook actuator (410) at the first detachment region (415). In some embodiments, the second grasping component (230) may comprise a prosthesis grasping hook. The prosthesis grasping hook may further comprise a prosthesis hook actuator (420) and a second detachment region (425). The prosthesis grasping hook may be actuated by the prosthesis hook actuator (420). Actuating the prosthesis hook actuator (420) may comprise pulling, twisting, pushing, or in any way moving the prosthesis hook actuator (420). The prosthesis grasping hook, upon anchoring to the prosthetic component (300), may detach from the prosthesis hook actuator (420) at the second detachment region (425).

In some embodiments, the chordae grasping hooks or the prosthesis grasping hooks are detachable. In some embodiments, the first detachment region (415) and the second detachment region (425) may also act as locks (e.g., quarter-turn locks).

In some embodiments, the delivery system (e.g., a catheter (100)) may guide the chordae grasping hooks or the prosthesis grasping hooks to mechanically turn the catheter (100) thus, in turn, turning the chordae grasping hooks or the prosthesis grasping hook. This may include twisting, pulling, pushing, rotating the catheter to grab the mitral leaflet component (e.g., the leaflets/chords). Once the chordae grasping hooks loop chordae tendineae (1), the chordae hook actuator (410) at the first detachment region (415) may be pulled back, thus pulling back the chordae grasping hooks into a secure position in the device.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A grasping device (200) for securing a leaflet component of a native mitral valve, wherein the leaflet component comprises a leaflet and chordae tendineae (1) associated with the leaflet, the device (200) comprising:
a) a first grasping component (210) configured to grab chordae tendineae (1) of said leaflet component of a native mitral valve; and
b) a second grasping component (230) configured to anchor the leaflet component to a prosthetic component (300).

2. The device (200) of claim 1, wherein the first grasping component (210) comprises a hook to grasp chordae.

3. The device (200) of claim 2, wherein the hook to grasp chordae further comprises a chordae hook actuator (410) and a first detachment region (415), wherein the hook to grasp chordae is actuated by the chordae hook actuator (410), wherein the hook to grasp chordae, upon grabbing chordae tendineae (1) of the leaflet component, detaches from the chordae hook actuator (410) at the first detachment region (415).

4. The device (200) of claim 1, wherein the second grasping component (230) comprises a hook to grasp the prosthesis.

5. The device (200) of claim 4, wherein the hook to grasp the prosthesis further comprises a prosthesis hook actuator (420) and a second detachment region (425), wherein the hook is actuated by the prosthesis hook actuator (420), wherein the hook to grasp the prosthesis, upon anchoring to the prosthetic component (300), detaches from the prosthesis hook actuator (420) at the second detachment region (425).

6. The device (200) of claim 1, wherein the first grasping component (210) comprises a grasping arm comprising a memory wire (215) disposed lengthwise and attached to a surface of the arm, wherein the arm is configured to transition from a non-extended state to an extended state to a grasping state, wherein transitioning from the extended state to the grasping state comprises pulling the memory wire (215) to curl the arm such that the first end meets or overlaps the second end of the arm.

7. The device (200) of claim 6 further comprising a first slide lock (220) adapted to slide between a locked position and an unlocked position along the device (200), wherein the unlocked position of the first slide lock (220) permits the arm to transition from the non-extended state to the extended state, wherein the locked position of the first slide lock (220) keeps the arm in the non-extended state or the grasping state.

8. The device (200) of claim 4, wherein the hook to grasp the prosthesis is disposed at a distal end of the device (200).

9. The device (200) of claim 8 further comprising a second slide lock (225) adapted to slide between a locked position and an unlocked position along the device (200), wherein the locked position closes the hook to grasp the prosthesis, and wherein the unlocked position allows the hook to grasp the prosthesis to anchor the native mitral leaflet component to the prosthetic component (300).

10. A method for securing a leaflet component of a native mitral valve, wherein the leaflet component comprises a leaflet and chordae tendineae (1) associated with the leaflet, the method comprising:
a) positioning a device (200) to a side of the chordae tendineae (1), said grasping device (200) comprising:
i) a first grasping component (210) configured to grab chordae tendineae (1) of said native mitral leaflet component; and
ii) a second grasping component (230) configured to anchor the leaflet component to a prosthetic component (300);
b) grabbing the chordae tendineae (1) using the first grasping component (210); and
c) anchoring the leaflet component to the prosthetic component (300) using the second grasping component (230).

11. The method of claim 10, wherein the first grasping component (210) comprises a hook to grasp chordae.

12. The method of claim 11, wherein the hook to grasp chordae further comprises a chordae hook actuator (410) and a first detachment region (415), wherein the hook to grasp chordae is actuated by the chordae hook actuator (410), wherein the hook to grasp chordae, upon grabbing chordae tendineae (1) of the mitral leaflet component, detaches from the chordae hook actuator (410) at the first detachment region (415).

13. The method of claim 10, wherein the second grasping component (230) comprises a hook to grasp the prosthesis.

14. The method of claim 13, wherein the hook to grasp the prosthesis further comprises a prosthesis hook actuator (420) and a second detachment region (425), wherein the hook to grasp the prosthesis is actuated by the prosthesis hook actuator (420), wherein the hook to grasp the prosthesis, upon anchoring to the prosthetic component (300), detaches from the prosthesis hook actuator (420) at the second detachment region (425).

15. The method of claim 10, wherein the first grasping component (210) of the device (200) comprises a grasping arm comprising a memory wire (215) disposed lengthwise and attached to a surface of the arm, wherein the grasping arm is configured to transition from a non-extended state to an extended state to a grasping state, wherein the method further comprises pulling the memory wire (215) to curl the arm such that the first end meets or overlaps the second end of the arm in order to transition from the extended state to the grasping state.

16. The method of claim 15, wherein the device (200) further comprises a first slide lock (220) adapted to slide between a locked position and an unlocked position along the device (200), wherein the unlocked position of the first slide lock (220) permits the arm to transition from the non-extended state to the extended state, wherein the locked position of the first slide lock (220) keeps the arm in the non-extended state or the grasping state.

17. The method of claim 16 further comprising:
a) sliding the first slide lock (220) into the unlocked position to permit an arm to transition from the non-extended state to the extended state; and
b) sliding the first slide lock (220) into the locked position to keep an arm in the grasping state.

18. The method of claim 13, wherein the hook to grasp the prosthesis is disposed at a distal end of the device (200).

19. The method of claim 18, wherein the device (200) further comprises a second slide lock (225) adapted to slide between a locked position and an unlocked position along the device (200), wherein the locked position closes the hook to grasp the prosthesis, and wherein the unlocked position allows the hook to grasp the prosthesis to anchor the native mitral leaflet component to the prosthetic component (300).

20. The method of claim 19 further comprising:
a) sliding the second slide lock (225) into the unlocked position to allow a hook to grasp the prosthesis to anchor the mitral leaflet component to the prosthetic component (300); and
b) sliding the second slide lock (225) into the locked position to lock the mitral leaflet component to the prosthetic component (300).
